(19) United States
(12) Patent Application Publication
(10) Pub. No.: US 2026/0123960 A1
(43) Pub. Date: May 7, 2026

(54) EXFOLIATION TOOL

(71) Applicant: Gerald McQuirter, Chicago, IL (US)

(72) Inventor: Gerald McQuirter, Chicago, IL (US)

(21) Appl. No.: 18/940,348

(22) Filed: Nov. 7, 2024

(51) Int. Cl. A61B 17/54; A61B 17/00

(52) U.S. Cl. CPC A61B 17/54; A61B 2017/00761

(57) ABSTRACT

An improved exfoliation tool is presented. The exfoliation tool is a substantially planar exfoliating face with a flexible central support structure having a top surface with an abrasive layer and a bottom surface attached to a plurality of adhesive discs for attachment to an angular surface of a device. The top surfaces of the plurality of chambers provide flexibility for the exfoliation tube and allow fluids to drain therefrom.

### FIELD OF THE INVENTION

The present invention refers to devices used to exfoliate the skin and more particularly to an improved exfoliation tool, which in its preferred embodiment, includes a flexible central support structure and a substantially planar exfoliating face allowing for angular foot exfoliation while the user is in a standing position.

### BACKGROUND OF THE INVENTION

Exfoliation tools for the feet are essential instruments in personal care, designed to remove coarse, flaky, parched, and dry skin that can accumulate due to various factors, including environmental conditions, footwear, and prolonged pressure on the feet. These tools are typically used to enhance foot hygiene, improve overall foot health, and provide aesthetic benefits by smoothing out rough patches and calluses.

Traditional foot exfoliation methods often involve the use of manual tools, such as pumice stones, foot scrubs, or abrasive files. While effective, these conventional methods can require significant effort and may not always provide optimal results, especially when users attempt to reach difficult areas. Furthermore, many existing exfoliating tools do not accommodate the natural posture of the user; necessitating bending or awkward positioning that can lead to discomfort or strain.

The typical operation of foot exfoliation involves the application of an abrasive surface to the foot, which is rubbed against the skin to remove dead skin cells. However, many tools lack features that facilitate fluid drainage, resulting in residue buildup and ineffective exfoliation. Additionally, most current designs do not leverage flexibility or ergonomic considerations, limiting their effectiveness and ease of use.

The present invention addresses these shortcomings by providing a foot care instrument that includes a uniquely constructed exfoliating tool. This tool features a flexible central support structure and a substantially planar exfoliating face, allowing for comfortable use while standing at various angles. The innovative arrangement of vertical and horizontal chambers on the tool's back enables effective fluid drainage, ensuring that the exfoliation process is both efficient and hygienic.

To this end, the novel and distinctive features of the disclosure consist of an exfoliation tool wherein the arrangement of the tool provides for a substantially planar exfoliating face allowing for angular foot exfoliation while the user is in a standing position. The exfoliation tool of the present disclosure is further characterized with the following novel features: (1) The substantially planar exfoliating face includes a flexible central support structure having a top surface with at least one abrasive layer covering the exfoliating face; and (2) The bottom surface of the central support structure is fixedly attached to a plurality of adhesive discs for attachment to the angular surface of a device and a plurality of uniformly sized and uniformly spaced vertical and horizontal chambers extending across the bottom surface of the central support structure.

### SUMMARY

This disclosure is an improved exfoliation tool having a substantially planar exfoliating face with a flexible central support structure. In an embodiment, the exfoliation tool further ccomprises the flexible central support structure having a top surface with at least one abrasive layer covering the exfoliating face and a bottom surface that is fixedly attached to a plurality of adhesive discs. The adhesive discs attach to the angular surface of a device and fixedly attach to the top surfaces of a plurality of chambers that extend across the top surface of the central support structure. This attachment allow fluids to drain through the exfoliation tool and allow for the angular placement of the exfoliation tool on the angular surface of a device.

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting. In case of conflict with terms used in the art, the present specification, including definitions, will control.

Additional advantages and features of the present invention will become more apparent when considered in light of the following specification and drawings.

### BRIEF DESCRIPTION OF THE DRAWINGS

The present embodiments are illustrated by way of the figures of the accompanying drawings, which may not necessarily be to scale, in which like references indicate similar elements, and in which:

FIG. 1 is a front view showing a foot exfoliator according to the present embodiment;

FIG. 2 is a left-side perspective view showing the raised appearance of the plurality of chambers and the adhesive discs;

FIG. 3 is a right-side perspective view showing the raised appearance of the plurality of chambers and the adhesive discs;

£ FIG. 4 is a top-side perspective view thereof;

FIG. 5 is a bottom-side perspective view thereof;

FIG. 6 is a back perspective view thereof showing the arrangement of the plurality of adhesive discs and chambers;

FIG. 7 is a perspective in-use view thereof showing the exfoliation tool of the present embodiment attached to the angular surface of a device;

FIG. 8 is a top view showing a foot exfoliator with the exfoliating face being removed from the central support structure;

FIG. 9 is a top view of the central support structure; and

FIG. 10 is a sectional view of an exfoliation tool of the present invention.

### DETAILED DESCRIPTION

The following detailed description is a contemplated mode of carrying out an exfoliation tool 10 and method of manufacture described herein. Although the exfoliation tool 10 and method of manufacture are explained in relation to an illustrated embodiment, it is understood that many possible modifications and variations can be made without departing from the spirit and scope of the disclosure.

Therefore, the exfoliation tool 10 contemplated by the disclosure is adapted to be used in various settings. It is desired that the exfoliation tool 10 include flexibility in design, easy modifications in structure, convenience in use, and efficiency in manufacturing. The inventor anticipates that all of the component parts of the completed exfoliation tool 10 will be properly aligned and that the component parts including the flexible central support structure 14, plurality of adhesive discs 24, abrasive layer 18, and chambers 32 will be securely attached and capable of effectively removing dry skin while allowing for proper drainage of fluid from the tool 10. User testing will be performed to confirm that the ergonomic design allows for the secured attachment to an angular surface of a device 26 and effective exfoliation. While a firm attachment of the abrasive layer 18 is preferred, the abrasive layer 18 can be designed to be easily removed from the top surface 16 of the central support structure 14 and therefore the surface can be further designed to be suitable for attaching, fastening, and detaching abrasives with an acceptable adhesive as required herein. In alternative embodiments, the various components including the central support structure 14 may be detachable from the abrasive layer 18, chambers 32, and discs 24 and may be easily replaced or the abrasive layer 18 and the central support structure 14 may be co-formed.

It can be appreciated that implementation of an exfoliation tool 10 and method of manufacture can include material for the surfaces of the central support structure 14 that are flexible, durable, and capable of horizontal and vertical arrangements. Flexible and durable material such as plastic, rubber, composite material, or the like can be used as the chamber material and all component parts. The most important requirement is that the material is durable and flexible enough to allow for the repeated use of the exfoliation tool 10 in a plurality of environments that permit for the angular placement and use of the same. The exfoliation tool 10 should maintain its durability while providing the user with a product that is practical and easy to manage.

It is anticipated that the sizes of the chamber material may vary according to the location of usage and style of the exfoliation tool 10 and the needs of a particular user. The chambers 32 may be constructed of many shapes and configurations including in the form of the rectangular rows and columns provided herein. These features enhance the user's experience while using the exfoliation tool 10. The water easily flows away from the exfoliation tool 10 and saturation of the central support structure 14 and the additional component parts is avoided. This ensures that the exfoliation tool 10 remains dry, minimizing the likelihood of mold and mildew development.

The material used for the central support structure 14 must be designed to withstand expansion from water pressure and to withstand the same to provide the flexibility effect on the bottom surface of the central support structure 14 thereof. The abrasive surface 18 provided along the top surface 16 of the central support structure 14 can be constructed from a an abrasive material so as to allow the desired exfoliation of the feet when moved in a back and forth manner thereon. The abrasive material can include various grits of sandpaper including coarser grits for tougher skin while finer grits can smoothen rough patches of skin. Synthetic abrasives, natural fibers, or the same can be used for milder abrasive action or as desired by a manufacuter or user.

The appealing features of the exfoliation tool 10 would be its functionality and the safety provided for the user. It may be sized to accommodate the foot allowing one foot to rest comfortably on the exfoliation tool 10. The appropriate size of the exfoliation tool 10 can be determined by considering the average dimensions of adult feet, ergonomic comfort, and ensuring enough surface area for effective exfoliation without compromising stability. The overall size, all parts, shapes, configuration, material composition, dimensions, and indicia applications would be developed during the design and engineering phases, prior to manufacture.

In an alternative embodiment of the foot exfoliation tool, the exfoliating face can include an adhesive layer designed for enhanced functionality. The bottom surface can further include an adhesive layer that is safeguarded by a peel-back layer. This adhesive layer can enable secure attachment of the exfoliation tool to the bottom and curved areas of a device such as a bathtub, ensuring stability during use. This design can further allow the bottom surface of the foot exfoliation tool to extend from the bathtub floor and curve upward along the tub's sides, facilitating comprehensive exfoliation of all areas of the user's feet. This alternative embodiment enhances the versatility of the foot exfoliation tool, making it further suitable for use in wet environments while maintaining the core benefits of exfoliation and skin care.

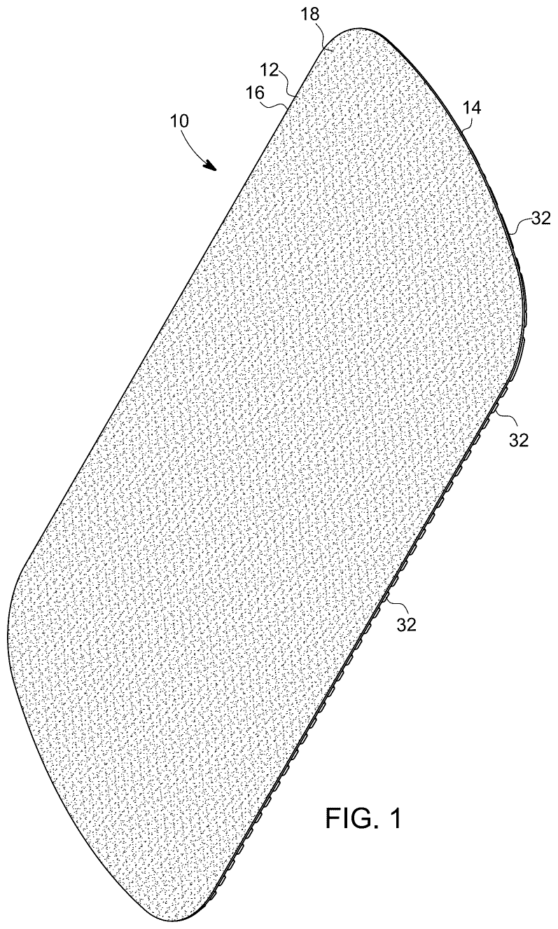

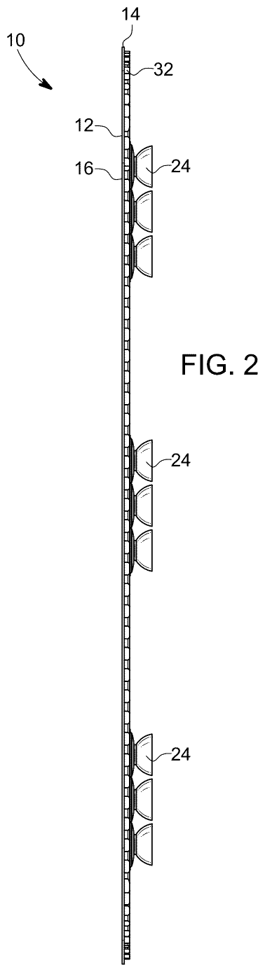

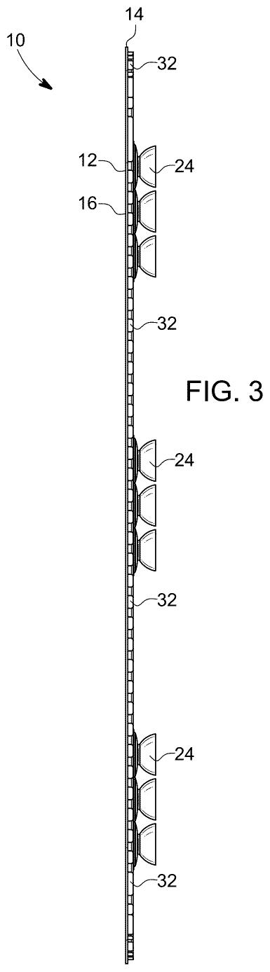

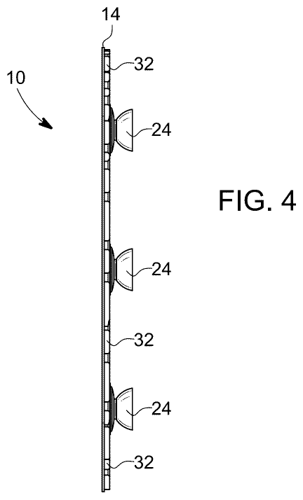

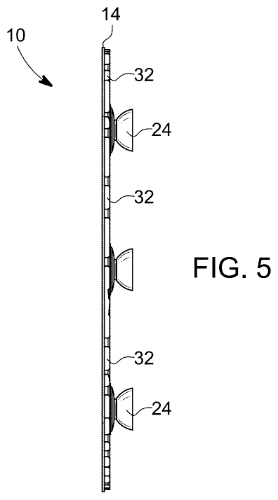

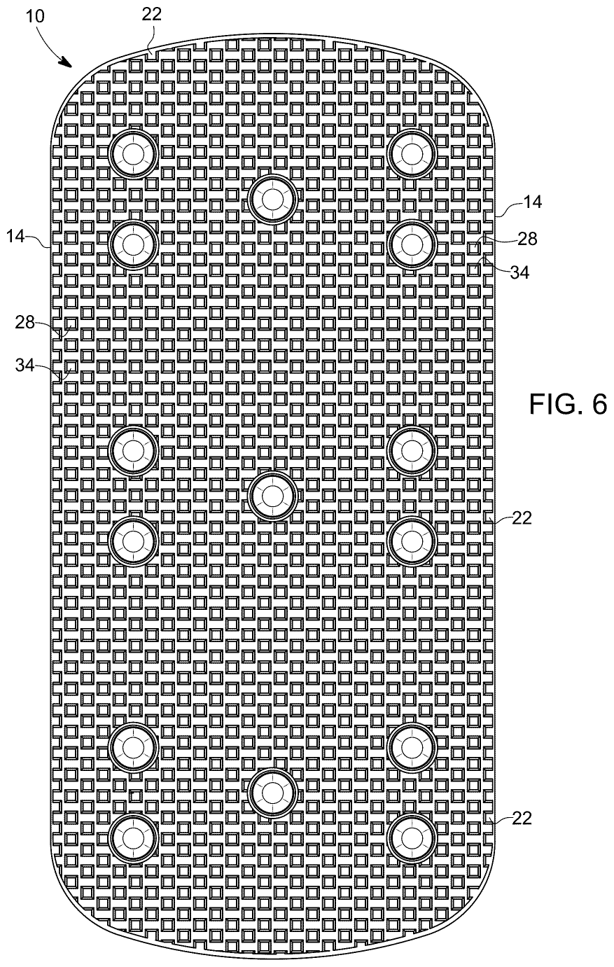

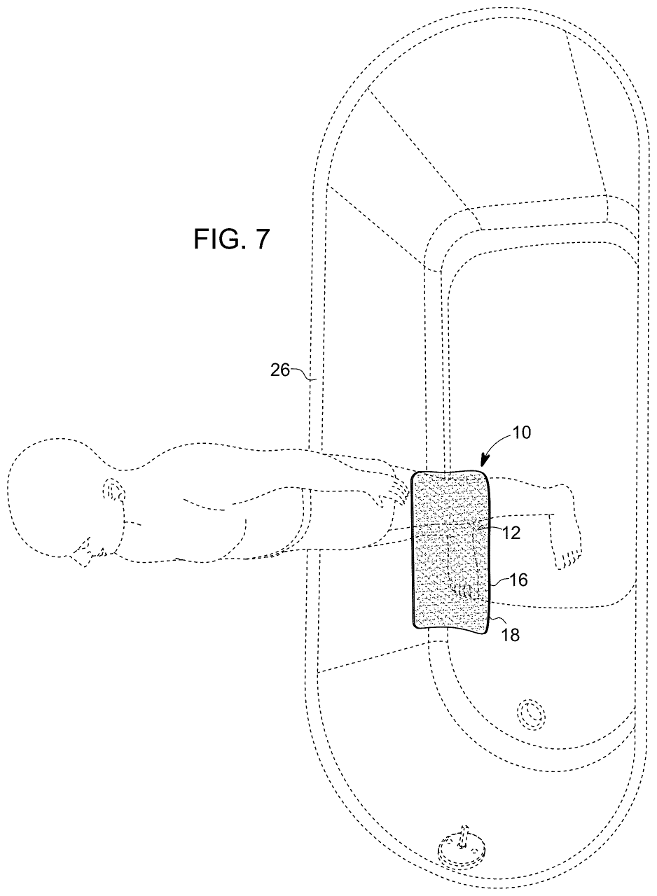

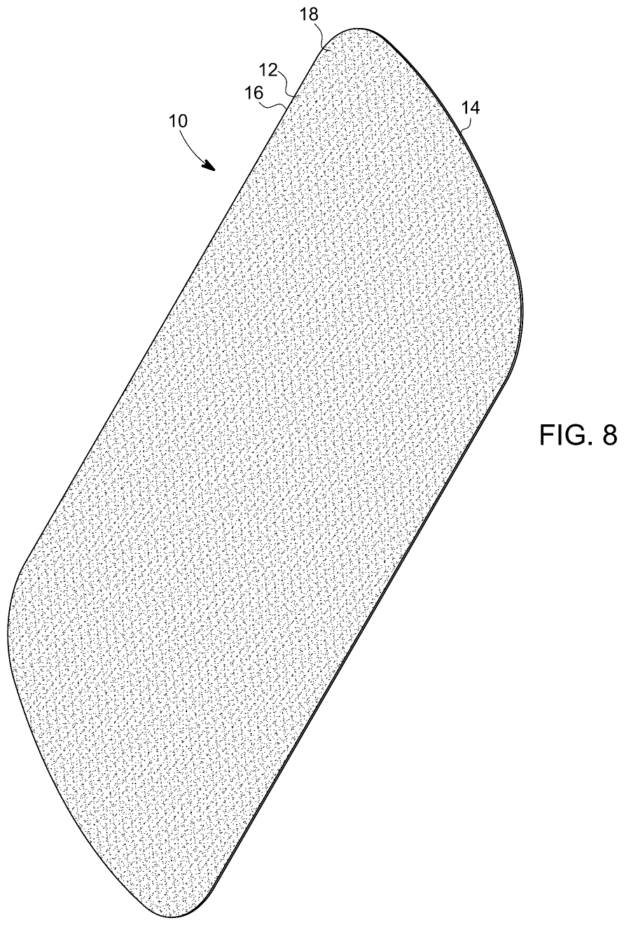

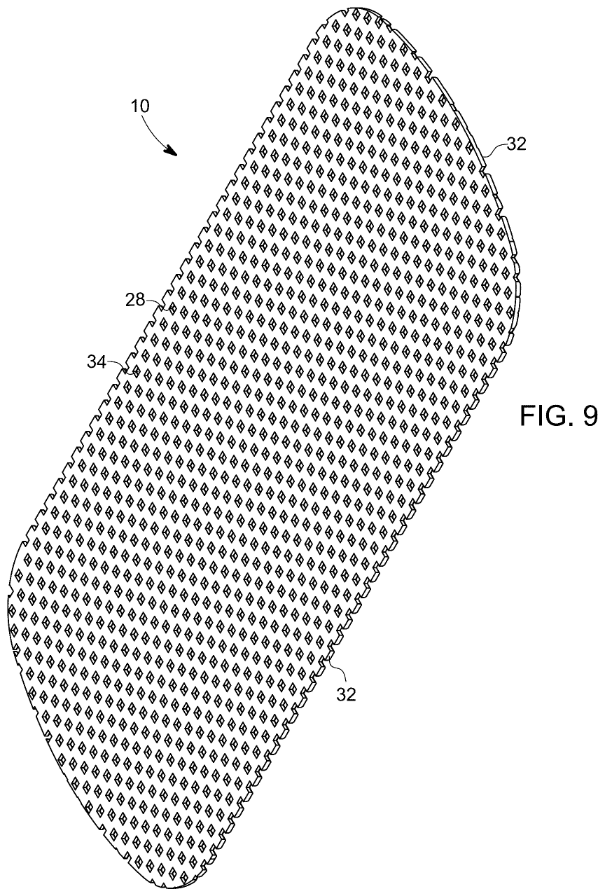

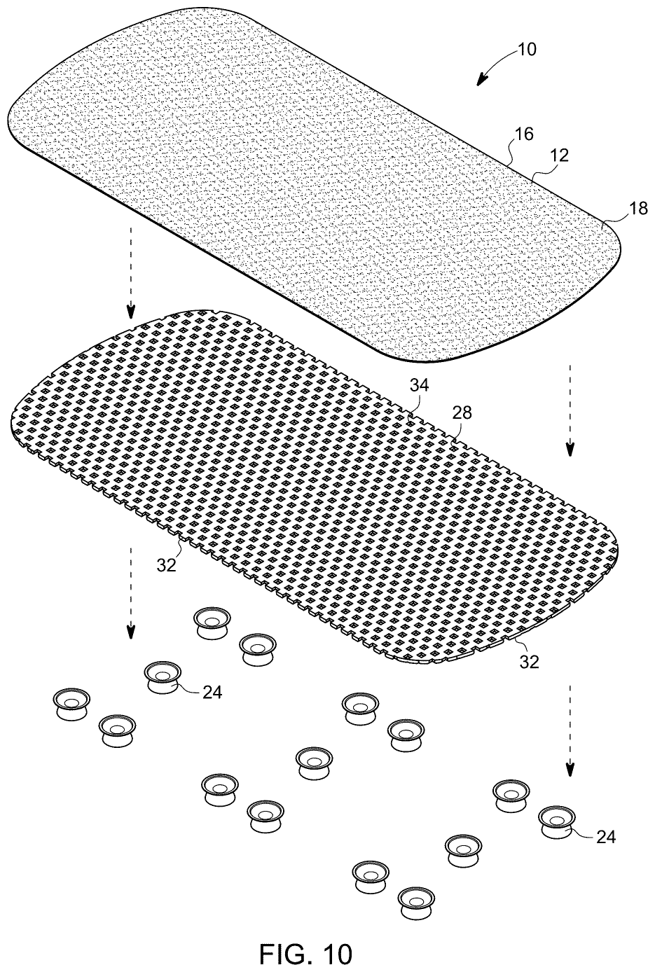

For example, and without limitation, FIGS. 1-9 show the exfoliation tool 10 according to one embodiment of the present disclosure wherein the exfoliation tool 10 is used to exfoliate the feet of a user. The exfoliation tool 10 comprising a substantially planar exfoliating face 12 with a flexible central support structure 14 having a top surface 16 with at least one abrasive layer 18 covering the exfoliating face 12 and the bottom surface. The bottom surface is fixedly attached to a plurality of adhesive discs 24 for attachment to an angular surface of a device 26 and fixedly attached to the top layers 28 of a plurality of uniformly sized and uniformly spaced vertical and horizontal chambers 32 having an interior 34 and being constructed of a flexible material. The chambers 32 extend across the bottom 22 surface 16 of the central support structure 14 to allow fluids to drain through the exfoliation tool 10 and for the angular placement and the removal of the exfoliation tool 10 from the angular surface of a device 26.

The central support structure 14 is designed to support at least partly a user while in a standing position. This convenient positioning allows a user to effortlessly and efficiently exfoliate the feet while maintaining a natural stance and minimizing tension on other areas of the body including the knees and the back. Caring for the feet can be challenging due to the formation of calluses and dry patches on various surfaces at different angles. Specifically, the heel is prone to developing rough skin on the uneven surfaces. The moist environment of devices such as a bathtubs improves the exfoliation experience, softening the skin for better results. This approach not only improves the efficacy of the exfoliation process but also transforms foot care into a relaxing routine. For example and without limitation, the exfoliation tool 10 of the present invention is preferably used on the angular surface of a device 26 such as a bath tub, shower, pedicure stations, workout stations, therapeutic pools, or the same where versatility and safety while exfoliating the feet are desired.

The discs 24 are thereby attachable to surfaces of ceramic tiles, glass, metal, plastic, wood, or the same that would ensure a secure and stable attachment during use. This ensures that the exfoliation tool 10 is securely attached to the surface, reduces the chances of slipping, and ensures a safer exfoliating experience. While mounted on the angular surface of a device 26 as provided herein, the exfoliation tool 10 is adjustable to any angle by turning and pressing the plurality of adhesive discs 24 into firm contact against curved areas of the surfaces 26 or against another angled surface 26 by detaching the tool 10 and readjusting a portion thereof to any portion of a surface 26.

An angular surface 26 for the devices described herein can be defined as a surface 26 that forms a specific geometric angle or slope, which facilitates the snug attachment of the exfoliation tool 10. This angular surface 26 allows for the integration of a bendable exfoliation tool 10 that can conform to the contours of available space provided on an angular surface 10 of a device. This design of the exfoliation tool 10 ensures stability and accessibility, enabling the exfoliation tool 10 to securely fit in place and allowing ease of use while standing in a moist environment.

The chambers 32 of the exfoliation tool 10 are disposed on the entirety of the bottom surface of the central support structure 14 and wherein fixedly attaching the bottom surface 22 of the central support structure 14 to the top layers 28 of the chamber 32 forms a top closure 36 for the chambers 28 and further allowing the chambers 32 to have a raised placement on the bottom surface 22 of the central support structure 14 in rows and columns.

Having described my invention, however, many modifications thereto will become apparent to those skilled in the art to which it pertains without deviation from the spirit of the invention as defined by the scope of the appended claims. Although the invention has been explained in relation to its preferred embodiment, it is to be understood that many other possible modifications and variations can be made without departing from the spirit and scope of the invention.

What is claimed is:

1. An exfoliation tool for exfoliating the feet of a user, the exfoliation tool comprising:
a substantially planar exfoliating face with a flexible central support structure having a top surface with at least one abrasive layer covering the exfoliating face; and
a bottom surface, wherein the bottom surface is fixedly attached to a plurality of adhesive discs for attachment to an angular surface of a device and fixedly attached to the top layers of a plurality of uniformly sized and uniformly spaced vertical and horizontal chambers having an interior and being constructed of a flexible material and extending across the top surface of the central support structure to allow fluids to drain through the exfoliation tool and for the angular placement and the removal of the exfoliation tool from the angular surface of a device.

2. The exfoliation tool according to claim 1 wherein the central support structure of the exfoliation tube is designed to support at least partly a user;

3. The exfoliation tool according to claim 1, wherein the chambers of the exfoliation tool are disposed on the entirety of the bottom surface of the central support structure.

4. The exfoliation tool according to claim 3, wherein fixedly attaching the bottom surface of the central support structure to the top layers of the chamber forms a top closure for the chambers.

5. The exfoliation tool according to claim 4, wherein the chambers are raised on the exterior surface and are arranged in rows and columns.

6. A method of construction for a an exfoliation tool, said method comprising the steps of:
selecting durable and flexible materials to create a substantially planar exfoliating face having a top surface and a bottom surface that are fixedly connected to create a flexible central support structure configured to at least partially support a user;
incorporating on the top surface of the exfoliating face at least one abrasive layer for foot exfoliation;
attaching a plurality of adhesive discs to the bottom surface of the central support structure;
designing and creating raised chambers on the exterior surface of the exfoliation tool, arranged in rows and columns, wherein the chambers are uniformly sized and spaced to facilitate fluid drainage;
creating the top closures of the chambers by fixedly attaching the bottom surface of the central support structure to the top layers of the chambers; fastening the inner layer to the outer layer to ensure a tight connection that allows for the formation of the chamber interiors;
fixedly attaching the outer layer of the exfoliation tool to the top layers of the chambers, thereby creating a plurality of uniformly sized and spaced vertical and horizontal chambers;
